(19) United States
(12) Patent Application Publication
(10) Pub. No.: US 2019/0130730 A1
(43) Pub. Date: May 2, 2019

(54) Alarm Management

(71) Applicant: COVIDIEN LP, Mansfield, MA (US)

(72) Inventor: ROBERT T. BOYER, Longmont, CO (US)

(21) Appl. No.: 15/993,412

(22) Filed: May 30, 2018

Related U.S. Application Data
(60) Provisional application No. 62/578,619, filed on Oct. 30, 2017.

(51) Int. Cl. G08B 25/00; G08B 21/18; G16H 40/67; G16H 10/60; A61B 5/00

(52) U.S. Cl. CPC G08B 25/001; G08B 21/182; A61B 5/0022; G16H 10/60; G16H 40/67

(57) ABSTRACT

Methods, systems, and devices for patient monitoring are described. The method may include measuring a physiological parameter associated with a patient and detecting an alarm condition based on the measured physiological parameter. After the alarm condition is detected, the medical device may receive an indication of medical data associated with the patient from a source other than the medical device. The method may further include determining whether to alarm according to the alarm condition and based on the received indication of medical data.

### CROSS REFERENCES

The present Application for Patent claims priority to U.S. Provisional Patent Application No. 62/578,619 by Boyer et al., entitled “ALARM MANAGEMENT”, filed Oct. 30, 2017, assigned to the assignee hereof.

### BACKGROUND

The following relates generally to patient monitoring, and more specifically to medical device alarm management.

In a healthcare facility such as a hospital, physiological parameters of a patient (e.g., heart rate, respiratory rate, blood pressure) may be monitored by one or more medical devices. A medical device is typically configured to sound an alarm or transmit an alarm notification once the measured physiological parameter exceeds or falls below a threshold. These thresholds may be statically configured by the device manufacturer or in some cases manually configured by a clinician. In any case, such medical devices are designed to sound alarms based on measured data at the device and transmit medical data and alarms in a unidirectional manner.

Because each medical device operates in its own information silo, if several medical devices associated with a patient detect a common physiological event, each may sound a different alarm or transmit a different alarm notification to a central nurse station. The multiple and simultaneous alarms from a patient may make it difficult for a clinician to discern relevant patient information and may cause unnecessary distraction. Also due to the information silo, medical devices make alarm decisions based on measured data at the device, regardless of other medically relevant information concerning the patient. As such, medical devices often trigger false alarms or may even fail to detect a significant physiological event. The increased number of false alarms may cause clinician alarm fatigue, which may lead to delayed responses that could put the patient at risk.

### SUMMARY

The described features generally relate to methods, systems, devices, or apparatuses that support alarm management. A medical device may detect an alarm condition associated with a physiological parameter of a patient. Before alarming in response to the alarm condition, the medical device may receive an indication of medical data associated with the patient from a source other than the medical device itself (e.g., medical data from other medical devices or manually input data). The indication may include instructions to suppress an alarm associated with the detected alarm condition, thereby overriding a default alarm behavior of the medical device. The indication may instead provide the medical device with additional medical information such that the medical device may be able to determine whether the detected alarm condition is actually indicative of a physiological decline and whether to alarm accordingly. In either case, the medical device may be configured with a fail-safe mode that triggers an alarm in response to the detected alarm condition if the indication of the additional medical data is delayed or is inconclusive.

A methods for patient monitoring is described. A method may include measuring, at a medical device, a physiological parameter associated with a patient. The method may also include detecting an alarm condition based at least in part on the measured physiological parameter. Additionally, the method may include receiving an indication of medical data associated with the patient from a source other than the medical device and determining whether to alarm according to the alarm condition based at least in part on the received indication.

Another apparatus for patient monitoring is described. The apparatus may include a processor, memory in electronic communication with the processor, and instructions stored in the memory. The instructions may be operable, when executed by the processor, to cause the apparatus to measure, at a medical device, a physiological parameter associated with a patient, detect an alarm condition based at least in part on the measured physiological parameter, receive an indication of medical data associated with the patient from a source other than the medical device, and determine whether to alarm according to the alarm condition based at least in part on the received indication.

A non-transitory computer readable medium for patient monitoring is described. The non-transitory computer-readable medium may include instructions executable by a processor to measure, at a medical device, a physiological parameter associated with a patient, detect an alarm condition based at least in part on the measured physiological parameter, receive an indication of medical data associated with the patient from a source other than the medical device, and determine whether to alarm according to the alarm condition based at least in part on the received indication.

Some examples of the method, apparatus, and non-transitory computer-readable medium described above may further include processes, features, or instructions for starting a fallback timer based at least in part on detecting the alarm condition. Some examples of the method, apparatus, and non-transitory computer-readable medium described above may further include processes, features, or instructions for alarming according to the alarm condition if the indication is not received before an expiration of the fallback timer. Additionally, some examples of the method, apparatus, and non-transitory computer-readable medium described above may further include processes, features, or instructions for transmitting a request to alarm based at least in part on detecting the alarm condition.

Some examples of the method, apparatus, and non-transitory computer-readable medium described above may further include processes, features, or instructions for receiving a decision message indicating to the medical device whether to alarm in response to the request. In some examples of the method, apparatus, and non-transitory computer-readable medium described above, the decision message is based at least in part on the medical data associated with the patient from the source other than the medical device. In some examples of the method, apparatus, and non-transitory computer-readable medium described above, the determining whether to alarm is further based at least in part on the decision message

Some examples of the method, apparatus, and non-transitory computer-readable medium described above may further include processes, features, or instructions for overriding a default alarm behavior associated with the alarm condition based at least in part on receiving the indication. Some examples of the method, apparatus, and non-transitory computer-readable medium described above may further include processes, features, or instructions for alarming based at least in part on receiving the indication. Additionally, some examples of the method, apparatus, and non-transitory computer-readable medium described above may further include processes, features, or instructions for transmitting a request for at least a subset of the medical data associated with the patient. In some examples of the method, apparatus, and non-transitory computer-readable medium described above, the indication of medical data is based at least in part on the request for at least the subset of the medical data.

In some examples of the method, apparatus, and non-transitory computer-readable medium described above, the source other than the medical device comprises one or more other medical devices, manually entered data, or both. In some examples of the method, apparatus, and non-transitory computer-readable medium described above, the determining whether to alarm is based at least in part on a priority of the medical device with respect to a priority of the source other than the medical device. In some examples of the method, apparatus, and non-transitory computer-readable medium described above, the alarm condition is a default alarm associated with the medical device.

Certain embodiments of the present disclosure may include some, all, or none of the above advantages or features. One or more other technical advantages or features may be readily apparent to those skilled in the art from the figures, descriptions, and claims included herein. Moreover, while specific advantages or features have been enumerated above, various embodiments may include all, some, or none of the enumerated advantages or features.

Further scope of the applicability of the described methods and systems will become apparent from the following detailed description, claims, and drawings. The detailed description and specific examples are given by way of illustration only, since various changes and modifications within the spirit and scope of the description will become apparent to those skilled in the art.

### BRIEF DESCRIPTION OF THE DRAWINGS

FIG. 1 illustrates an example of a system for patient monitoring that supports alarm management in accordance with aspects of the present disclosure.

FIG. 2 illustrates an example of a patient monitoring system that supports alarm management in accordance with aspects of the present disclosure.

FIG. 3 illustrates an example process flow that supports alarm management in accordance with aspects of the present disclosure.

FIGS. 4 through 6 show block diagrams of a device that supports alarm management in accordance with aspects of the present disclosure.

FIG. 7 illustrates a block diagram of a system including a medical device that supports alarm management in accordance with aspects of the present disclosure.

FIGS. 8 through 11 illustrate methods for alarm management in accordance with aspects of the present disclosure.

### DETAILED DESCRIPTION

In a healthcare facility, one or more alarms from a patient may be sounding at a central nurse station, each of which may pertain to different information of the patient. The one or more alarms may be sound from a variety of different medical devices used to monitor the patient. In some cases, multiple medical devices may be used to monitor a single patient, each of which may sound an alarm based on certain criteria. For example, a medical device may be used to monitor the heart rate of a patient and may sound when the heart rate falls below a minimum threshold. Another medical device may be used to monitor the respiratory rate of the patient and may sound when the respiratory rate reaches a maximum threshold.

When multiple alarms are being sound from multiple medical devices monitoring a patient, the clinician(s) responsible for the patient may be unable to determine which alarm requires immediate attention. In some cases, the multiple alarms may be interrelated with multiple medical devices. However, by managing the alarms in accordance with aspects of the present disclosure, a clinician or group of clinicians monitoring the patient may be able to discern the alarm of interest and tend to the patient more quickly and accurately.

In some cases, the medical device may trigger a false alarm. For example, the medical device may not detect an actual physiological event associated with the measured parameter of the patient, but an outside source (e.g., movement of the patient, measurements performed by another medical device, etc.) may cause the medical device to alarm. In that case, the medical device may request data from the outside source to determine whether to alarm. Therefore, medical devices may communicate bidirectionally to prevent false alarms. Based on the requested data, the medical device may suppress the false alarm or continue to alarm (e.g., indicating a real alarm).

For example, the patient may be monitored by a medical device (e.g., a pulse oximeter) that measures the oxygen concentration in the blood and sounds when the oxygen concentration falls below a certain threshold. However, the clinician may also connect the patient to a blood pressure monitor and place an inflatable cuff on the patient to determine the patient's blood pressure. As the cuff inflates, the blood pressure of the patient increases and the oxygen concentration in the blood decreases. In some cases, the pulse oximeter may request information from the blood pressure monitor to determine whether to alarm based on exceeding a threshold. Therefore, the pulse oximeter and the blood pressure monitor may communicate with each other to manage the alarms of the respective medical devices. The blood pressure monitor may then communicate the requested information to the pulse oximeter, where the pulse oximeter may determine to suppress an alarm.

In some cases, multiple medical devices monitoring a patient may share medical information associated with the patient. Therefore, the information silo of the respective medical devices may be broken down to enable the transmission of information between several medical devices. When multiple medical devices detect the same patient condition and corresponding alarm condition, a single medical device may alarm. That is, the other medical devices associated with the measured physiological parameter may suppress an alarm in response to a received signal from the alarming medical device or a central station. Therefore, duplicative alarms may be prevented, and the effort of the clinician to turn off multiple alarms may be reduced. In some cases, preventing duplicative alarms may also reduce the number of false alarms.

In some cases, a central server may aggregate the information received from one or more medical devices to send a message of whether to alarm. For example, the central server may communicate a message to annunciate or suppress an alarm associated with the medical device. In some examples, the medical device may request to alarm and include a delay threshold, after which the delay threshold is exceeded and a lack of response may result in a fallback alarm.

Aspects of the disclosure are initially described in the context of a wireless patient monitoring system. Aspects of the disclosure are further illustrated by and described with reference to apparatus diagrams, system diagrams, and flowcharts that relate to alarm management.

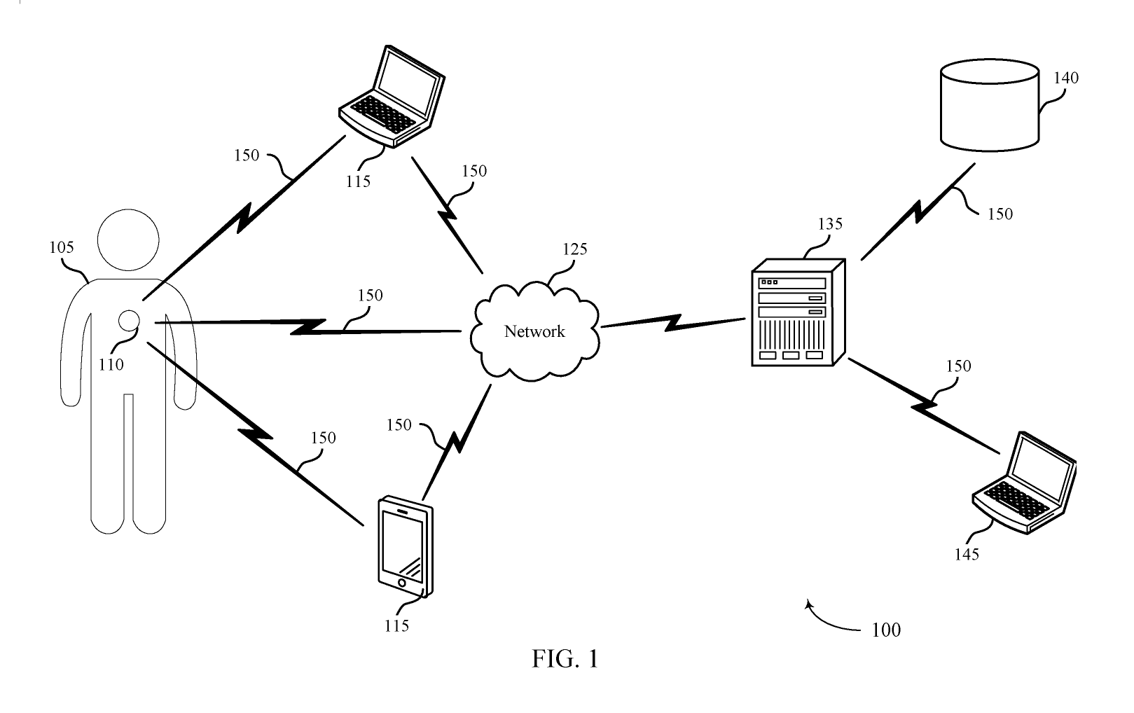

FIG. 1 illustrates an example of a patient monitoring system 100 in accordance with various embodiments of the present disclosure. The patient monitoring system 100 may include a patient 105 wearing, carrying, or otherwise coupled with a medical device 110. Although a single medical device 110 is shown, multiple medical devices 110 may be coupled to the patient 105. The patient 105 may be a patient in a hospital, nursing home, home care, a medical facility, or another care facility. The medical device 110 may transmit signals via wireless or wired communications links 150 to computing devices 115 or to a network 125.

The medical device 110 may include one or more sensors configured to collect a variety of physiological parameters as well as information related to the location and movement of the patient 105. For example, the medical device 110 may include a pulse oximetry (SpO2) sensor, a capnography sensor, a heart rate sensor, a blood pressure sensor, an electrocardiogram (ECG) sensor, a respiratory rate sensor, a glucose level sensor, a depth of consciousness sensor, a body temperature sensor, an accelerometer, a global positioning sensor, a sensor which triangulates position from multiple computing devices 115, or any other sensor configured to collect physiological, location, or motion data associated with the patient 105.

The medical device 110 may be coupled with the patient 105 in a variety of ways depending on the data being collected. For example, the medical device 110 may be directly coupled with the patient 105 (e.g., physically connected to the patient's chest, worn around the patient's wrist, attached to the patient's finger, or positioned over the patients nose or mouth). The data collected by the medical device 110 may be transmitted to either the computing devices 115 or to the remote computing device 145 (via the network 125 and central station 135). Wireless data transmission may occur via, for example, frequencies appropriate for a personal area network (such as Bluetooth, Bluetooth Low Energy (BLE), or IR communications) or local (e.g., wireless local area network (WLAN)) or wide area network (WAN) frequencies such as radio frequencies specified by IEEE standards (e.g., IEEE 802.15.4 standard, IEEE 802.11 standard (Wi-Fi), IEEE 802.16 standard (WiMAX), or cellular or satellite communications, or any other wireless communication type. Wired data transmissions may occur over any wired connections using any communication protocol (e.g., Ethernet). System communications may use one or both wired and wireless data transmissions.

Computing devices 115 may be examples of a wireless device such as a tablet, cellular phone, personal digital assistant (PDA), a dedicated receiver, or other similar device or a spatially distributed network of devices configured to receive signals from the medical device 110. In some cases, the computing devices 115 may be a wireless laptop computer, a clinician Workstation on Wheels, or a smart hospital bed configured to receive signals from the medical device 110. The computing devices 115 may be in communication with a central station 135 via network 125.

The medical device 110 may also communicate directly with the central station 135 via the network 125. The central station 135 may be a server or a central nurse station located within the hospital or in a remote location. The central station 135 may be in further communication with one or more remote computing devices 145, thereby allowing a clinician to remotely monitor the patient 105. The central station 135 may also be in communication with various remote databases 140 where the collected patient data may be stored or from which patient data may be retrieved. In some cases, the remote databases 140 include electronic medical records (EMR) applications for storing and sharing patient data.

In accordance with aspects of the present disclosure, the medical device 110 may detect an alarm condition based on measuring a physiological parameter associated with the patient 105. For example, the medical device 110 may be measuring the heart rate of the patient 105 and may detect that the heart rate of the patient 105 has exceeded an alarm threshold. Before sounding an alarm in response to the detected alarm condition, the medical device 110 may receive an indication of medical data associated with the patient 105 from a source other than the medical device 110 and may determine whether to alarm according to the detected alarm condition based on the received indication.

For example, the medical device 110 may receive the indication from the central station 135, a computing device 115, or from a different medical device 110 associated with the patient 105, and the source of the medical data may be the different medical devices 110 or manually input data at the central station 135. As described in more detail below, the received indication of medical data associated with the patient 105 may include instructions for the medical device 110 to either suppress or annunciate an alarm, or may instead provide the medical device 110 with additional medical data information such that the medical device 110 can determine whether to alarm itself. Such an indication may help reduce the instances of false alarms, as the medical device 110 may be able to aggregate medically relevant data for the patient 105 to determine if a detected alarm condition is actually indicative of a physiological event. Similarly, in cases of an actual physiological event, such an indication may suppress the alarms of certain medical devices 110 so that several medical devices 110 are not simultaneously alarming in response to a single medical event.

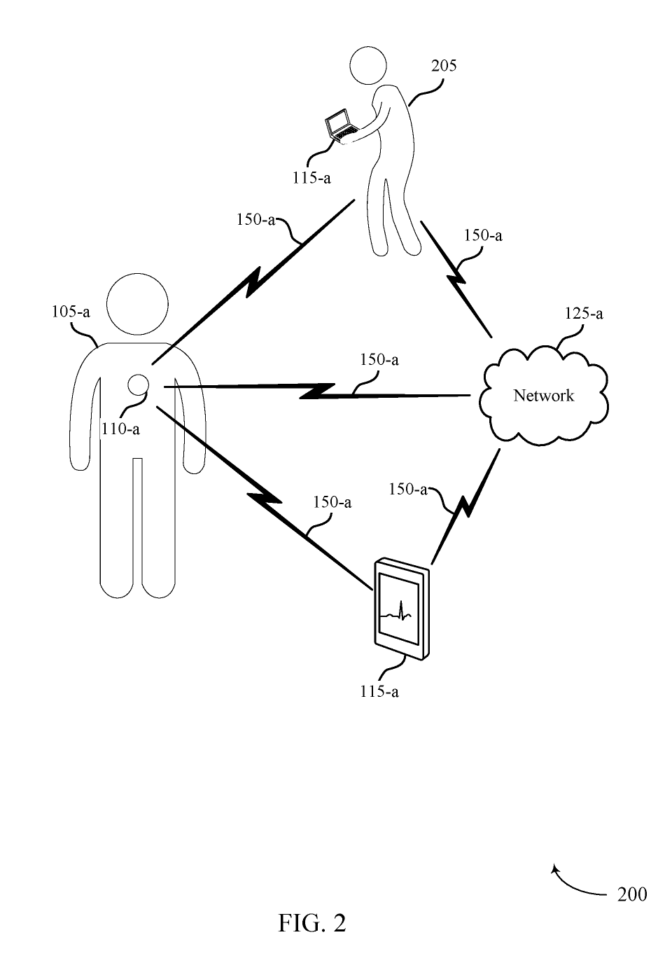

FIG. 2 illustrates an example of a patient monitoring system 200 that supports alarm management in accordance with aspects of the present disclosure. The patient monitoring system 200 may be an example of aspects of patient monitoring system 100 and may include a patient 105-a wearing, carrying, or otherwise coupled with a medical device 110-a. The medical device 110-a may include one or more sensors configured to measure a variety of physiological parameters associated with the patient 105-a. Medical device 110-a may also detect an alarm condition associated with a measured physiological parameter. The alarm condition may be based on default parameter thresholds that are configured by the manufacturer of the medical device 110-a or that are manually configured by a clinician 205.

The medical device 110-a may communicate bidirectionally via wired or wireless communication links 150-a to computing devices 115-a or to network 125-a. Computing devices 115-a may be an example of another medical device measuring a physiological parameter of the patient 105-a. The computing devices 115-a may also be an example of a device that accepts and stores manually input medical data associated with the patient 105-a that is manually input from a clinician 205. For example, the manually input data may include lab results for the patient 105-a or general medical information such as weight, height, age, gender, previous or current medical conditions, currently prescribed medications, or similar medical information that may not be generally associated with a medical device 110-a monitoring the patient 105-a. Additionally or alternatively, the computing devices 115-a may be an example of a central station or server that aggregates medical data associated with the patient 105-a from multiple medical devices or manually input data. In each case, the computing devices 115-a may be an example of a source of medical data associated with the patient 105-a that is different from the medical device 110-a. As described in more detail below, the medical device 110-a may receive an indication of medical data associated with the patient 105-a from one or more of these computing devices 115-a, and the medical device 110-a may determine whether to alarm based on the received indication.

In some examples, the medical device 110-a may transmit a request or a desire to alarm via wireless communication links 150-a in response to detecting an alarm condition. In some cases, medical device 110-a may receive feedback from computing devices 115-a or network 125 to suppress an alarm associated with the detected alarm condition. This feedback may be based on medically relevant information that is collected by the computing devices 115-a or the network 125-a. For example, network 125-a may receive data (e.g., sensor data or manually input data) from a source other than medical device 110-a (e.g., computing devices 115-a or other medical sensors), and this received data may indicate that the detected alarm condition at the medical device 110-a is actually a false alarm. The computing devices 115-a or network 125-a may then send an indication to the medical device 110-a of this false alarm by instructing the medical device 110-a to suppress its alarm. As such, the default alarm protocol or behavior associated with medical device 110-a may be overridden based on the received feedback.

In some cases, patient monitoring system 200 may support a publish-to-subscribe operation. For example, medical device 110-a may collect information from multiple sources (e.g., other sensors associated with the patient 105-a ) and may determine whether to alarm based on the collected information. In some cases, medical device 110-a may determine to sound an alarm even though the medical device 110-a may not detect an alarm condition based on its default alarm thresholds. That is, the information received at medical device 110-a from an outside source may cause medical device 110-a to alarm. For example, even if a physiological parameter being measured by the medical device 110-a appears to be within normal range, the medical device 110-a may still decide to alarm based on other received medical data that, combined with the measured data at the medical device 110-a, indicates an imminent physiological event.

In some cases, medical device 110-a may alarm according to a fallback or failsafe alarm configuration. For example, if the medical device 110-a detects an alarm condition and transmits a request or desire to alarm, the medical device 110-a may alarm according to the detected alarm condition if the medical device 110-a does not receive an alarm decision in response to the alarm request during a predetermined time interval. The predetermined time interval may be measured by a fallback timer that starts once the alarm condition is detected or once the alarm request is sent. For example, the fallback alarm mode may activate when the network communication is unavailable or the network communication is slow. In some cases, the predetermined time interval may be based on the type of medical device 110-a or the severity of the physiological parameter measured. For example, a ventilator may alarm after a shorter predetermined time interval expires as compared to a pulse oximeter.

Medical device 110-a may also alarm based on a priority preference. For example, medical device 110-a may alarm based on the severity of the physiological parameter measured by medical device 110-a or the significance of the medical device 110-a. The severity of the physiological parameter may be associated with the number of medical devices 110-a alarming or the type of medical device 110-a alarming. Medical device 110-a may also alarm in response to patient 105-a exceeding an alarm threshold for a physiological parameter associated with patient 105-a. In response, medical device 110-a may then transmit a signal via wireless communication links 150-a to network 125-a to notify clinician 205.

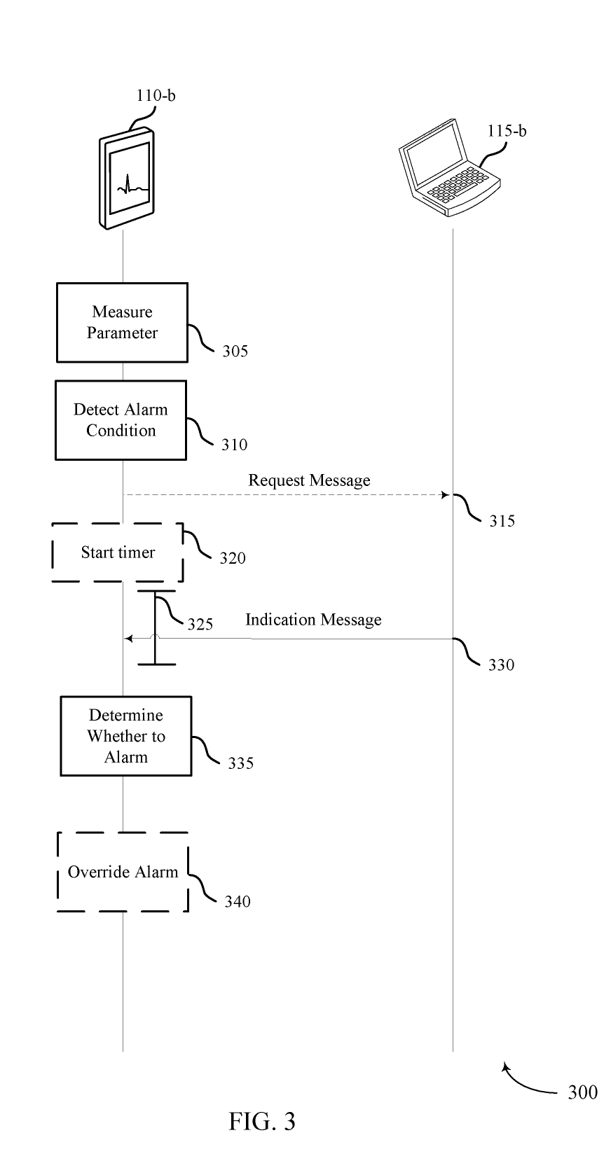

FIG. 3 illustrates an example process flow 300 that supports alarm management in accordance with aspects of the present disclosure. Process flow 300 may include medical monitoring device 110-b and source 115-b, which may be respective examples of a medical device 110 and computing device 115 as described with reference to FIGS. 1 and 2. Source 115-b may be one or more other medical devices, manually entered data, or both. Alternative examples of the following may be implemented, where some steps are performed in a different order or not at all. Some steps may additionally include additional features not mentioned above.

At block 305, medical monitoring device 110-b may measure a physiological parameter associated with a patient. At block 310, medical monitoring device 110-b may detect an alarm condition based on the measured physiological parameter.

After the alarm condition is detected, medical monitoring device 110-b may transmit request message 315. In some examples, request message 315 may be a request or a desire to alarm based on detecting the alarm condition. In other examples, request message 315 may be a request for medical data. For example, medical monitoring device 110-b may subscribe to medical data from a source other than medical monitoring device 110-b in a published network (e.g., publish-to-subscribe operation). Request message 315 may be for at least a subset of the medical data associated with the patient. In some cases, request message 315 may transmit information related to a physiological parameter associated with the patient. In such cases, the receiver of the request message 315 may utilize the information to make a decision regarding whether to alarm.

At block 320, medical monitoring device 110-b may start a timer. For example, the timer may be a fallback or failsafe timer. In some examples, starting the fallback timer may be based on detecting the alarm condition. Fallback timer may start after medical monitoring device 110-b transmits request message 315. In some cases, fallback timer may start at the same time that medical monitoring device 110-b transmits request message 315. The fallback timer may extend for duration 325. Duration 325 may be for a predetermined time interval based on the alarm threshold of medical monitoring device 110-b.

Source 115-b may transmit indication message 330. In some cases, indication message 330 may indicate to medical monitoring device 110-b whether to alarm in response to request message 315 (e.g., an alarm decision message). In some examples, indication message 330 may include a subset of medical data based on request message 315. Indication message 330 may be based on the medical data associated with the patient from the source other than medical monitoring device 110-b (e.g., source 115-b). In some cases, medical monitoring device 110-b may use the medical data of indication message 330 to determine whether to alarm.

If medical monitoring device 110-b receives indication message 330 before duration 325 expires, medical monitoring device 110-b may determine whether to alarm based on indication message 330. If duration 325 expires before medical monitoring device 110-b receives indication message 330, medical monitoring device 110-b may alarm based on a fallback or failsafe mode.

At block 335, medical monitoring device 110-b may determine whether to alarm based on indication message 330. For example, medical monitoring device 110-b may determine to alarm. In some examples, determining whether to alarm may be based at least in part on a priority of medical monitoring device 110-b with respect to a priority of the source other than the medical device (e.g., source 115-b). At block 340, medical monitoring device 110-b may override the alarm (e.g., not sound an alarm even though the default alarm threshold indicates an alarm should be sound). In some examples, overriding a default alarm behavior associated with the alarm condition may be based on receiving indication message 330.

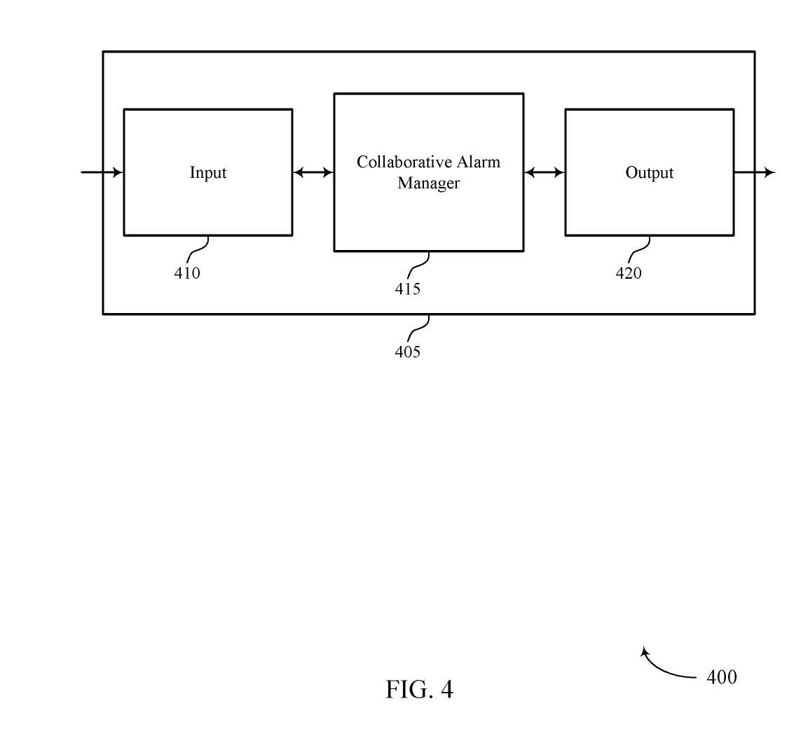

FIG. 4 shows a block diagram 400 of a device 405 that supports alarm management in accordance with aspects of the present disclosure. Device 405 may be an example of aspects of a medical device as described herein. Device 405 may include input 410, collaborative alarm manager 415, and output 420. Device 405 may also include a processor. Each of these components may be in communication with one another (e.g., via one or more buses).

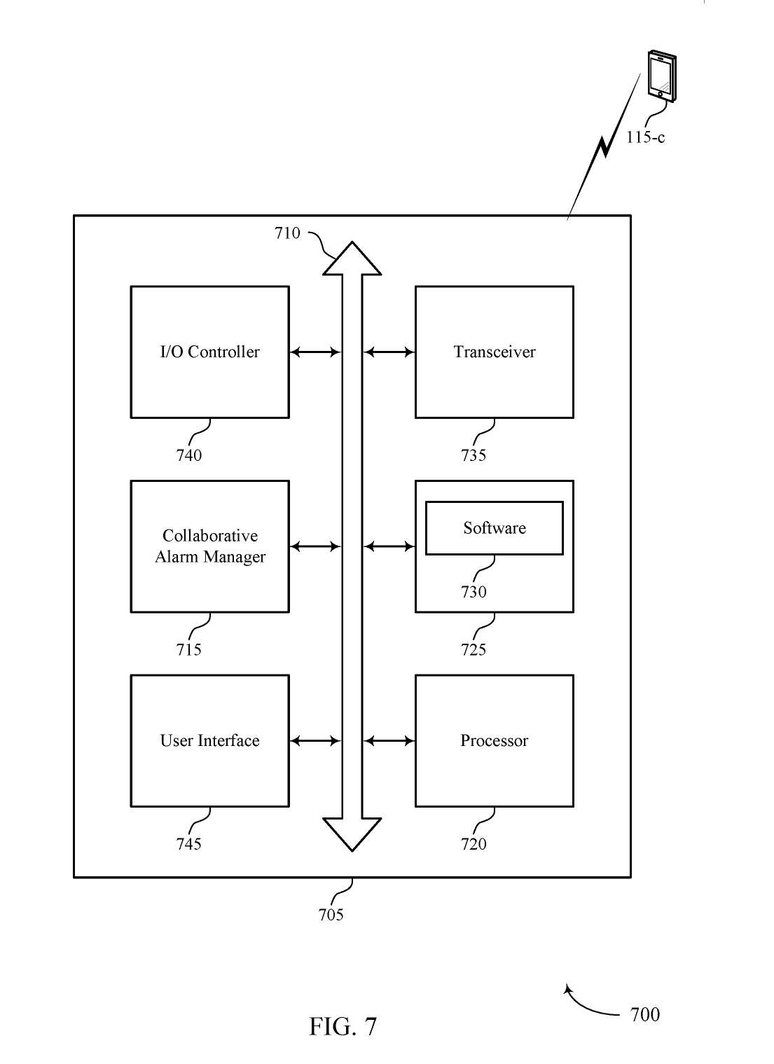

Collaborative alarm manager 415 may be an example of aspects of the collaborative alarm manager 715 described with reference to FIG. 7.

Collaborative alarm manager 415 and/or at least some of its various sub-components may be implemented in hardware, software executed by a processor, firmware, or any combination thereof. If implemented in software executed by a processor, the functions of the collaborative alarm manager 415 and/or at least some of its various sub-components may be executed by a general-purpose processor, a digital signal processor (DSP), an application-specific integrated circuit (ASIC), an field-programmable gate array (FPGA) or other programmable logic device, discrete gate or transistor logic, discrete hardware components, or any combination thereof designed to perform the functions described in the present disclosure. The collaborative alarm manager 415 and/or at least some of its various sub-components may be physically located at various positions, including being distributed such that portions of functions are implemented at different physical locations by one or more physical devices. In some examples, collaborative alarm manager 415 and/or at least some of its various sub-components may be a separate and distinct component in accordance with various aspects of the present disclosure. In other examples, collaborative alarm manager 415 and/or at least some of its various sub-components may be combined with one or more other hardware components, including but not limited to an I/O component, a transceiver, a network server, another computing device, one or more other components described in the present disclosure, or a combination thereof in accordance with various aspects of the present disclosure.

Collaborative alarm manager 415 may measure, at a medical device, a physiological parameter associated with a patient, detect an alarm condition based on the measured physiological parameter, receive an indication of medical data associated with the patient from a source other than the medical device, and determine whether to alarm according to the alarm condition based on the received indication.

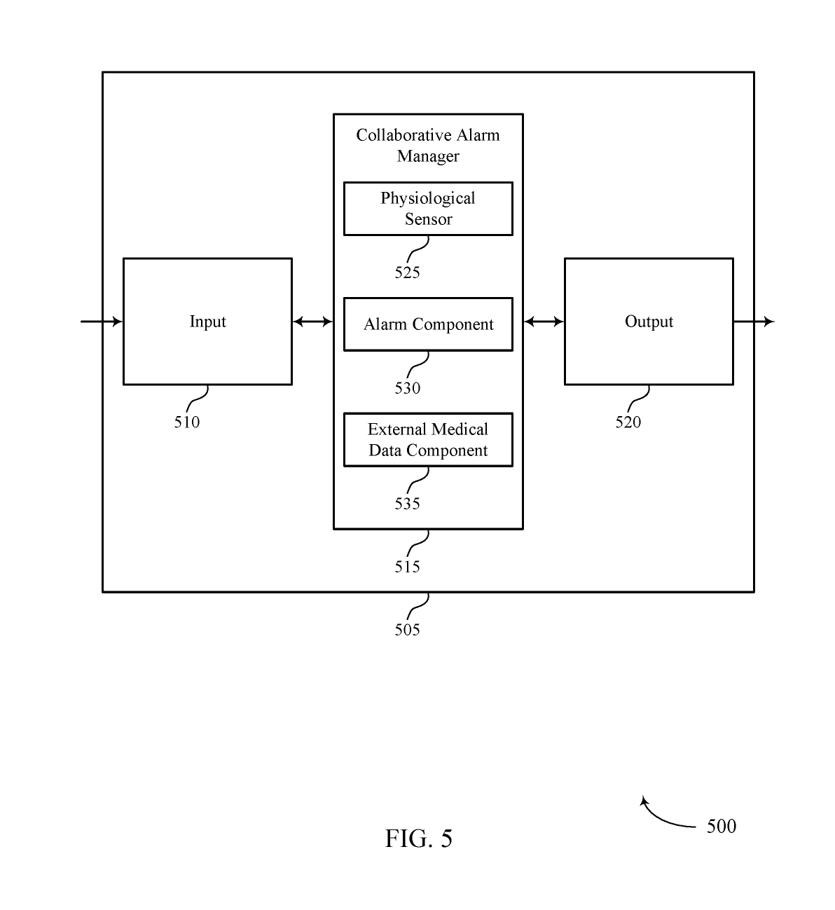

FIG. 5 shows a block diagram 500 of a device 505 that supports alarm management in accordance with aspects of the present disclosure. Device 505 may be an example of aspects of a device 405 or a medical device as described with reference to FIG. 4. Device 505 may include input 510, collaborative alarm manager 515, and output 520. Device 505 may also include a processor. Each of these components may be in communication with one another (e.g., via one or more buses).

Collaborative alarm manager 515 may be an example of aspects of the collaborative alarm manager 715 described with reference to FIG. 7.

Collaborative alarm manager 515 may also include physiological sensor 525, alarm component 530, and external medical data component 535.

Physiological sensor 525 may measure, at a medical device, a physiological parameter associated with a patient.

Alarm component 530 may detect an alarm condition based on the measured physiological parameter and determine whether to alarm according to the alarm condition based on the received indication. Alarm component 530 may also alarm according to the alarm condition if the indication is not received before an expiration of the fallback timer, override a default alarm behavior associated with the alarm condition based on receiving the indication, and alarm based on receiving the indication. In some cases, determining whether to alarm is based on a priority of the medical device with respect to a priority of the source other than the medical device. In some cases, the alarm condition is a default alarm associated with the medical device.

External medical data component 535 may receive an indication of medical data associated with the patient from a source other than the medical device. In some cases, the source other than the medical device includes one or more other medical devices, manually entered data, or both.

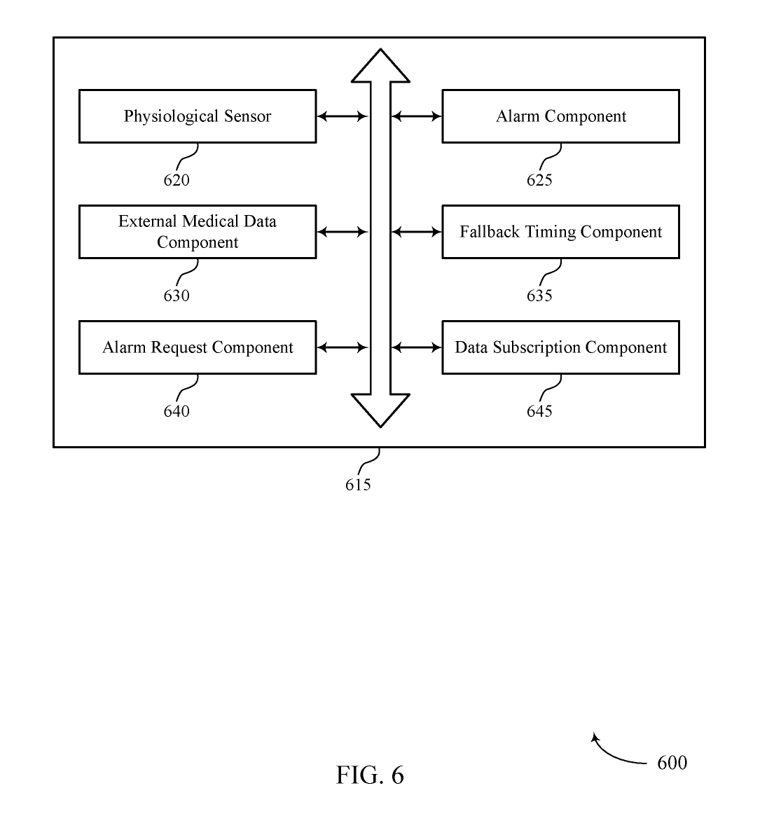

FIG. 6 shows a block diagram 600 of a collaborative alarm manager 615 that supports alarm management in accordance with aspects of the present disclosure. The collaborative alarm manager 615 may be an example of aspects of a collaborative alarm manager 415 or a collaborative alarm manager 515 described with reference to FIGS. 4 and 5. The collaborative alarm manager 615 may include physiological sensor 620, alarm component 625, external medical data component 630, fallback timing component 635, alarm request component 640, and data subscription component 645. Each of these modules may communicate, directly or indirectly, with one another (e.g., via one or more buses).

Physiological sensor 620 may measure, at a medical device, a physiological parameter associated with a patient.

Alarm component 625 may detect an alarm condition based on the measured physiological parameter and determine whether to alarm according to the alarm condition based on the received indication. Alarm component 625 may alarm according to the alarm condition if the indication is not received before an expiration of the fallback timer, override a default alarm behavior associated with the alarm condition based on receiving the indication, and alarm based on receiving the indication. In some cases, the determining whether to alarm is based on a priority of the medical device with respect to a priority of the source other than the medical device. In some cases, the alarm condition is a default alarm associated with the medical device.

External medical data component 630 may receive an indication of medical data associated with the patient from a source other than the medical device. In some cases, the source other than the medical device includes one or more other medical devices, manually entered data, or both.

Fallback timing component 635 may start a fallback timer based on detecting the alarm condition.

Alarm request component 640 may transmit a request to alarm based on detecting the alarm condition and receive a decision message indicating to the medical device whether to alarm in response to the request, where the decision message is based on the medical data associated with the patient from the source other than the medical device, and where the determining whether to alarm is further based on the decision message.

Data subscription component 645 may transmit a request for at least a subset of the medical data associated with the patient, where the indication of medical data is based on the request for at least the subset of the medical data.

FIG. 7 shows a diagram of a system 700 including a device 705 that supports alarm management in accordance with aspects of the present disclosure. Device 705 may be an example of or include the components of device 405, device 505, or a medical device as described above, e.g., with reference to FIGS. 4 and 5. Device 705 may include components for bi-directional voice and data communications including components for transmitting and receiving communications, including collaborative alarm manager 715, processor 720, memory 725, software 730, transceiver 735, I/O controller 740, and user interface 745. These components may be in electronic communication via one or more buses (e.g., bus 710).

Processor 720 may include an intelligent hardware device, (e.g., a general-purpose processor, a DSP, a central processing unit (CPU), a microcontroller, an ASIC, an FPGA, a programmable logic device, a discrete gate or transistor logic component, a discrete hardware component, or any combination thereof). The processor 720 may process information received from computing device 115-c. In some cases, processor 720 may be configured to operate a memory array using a memory controller. In other cases, a memory controller may be integrated into processor 720. Processor 720 may be configured to execute computer-readable instructions stored in a memory to perform various functions (e.g., functions or tasks supporting alarm management).

Memory 725 may include random access memory (RAM) and read only memory (ROM). The memory 725 may store computer-readable, computer-executable software 730 including instructions that, when executed, cause the processor to perform various functions described herein. In some cases, the memory 725 may contain, among other things, a basic input/output system (BIOS) which may control basic hardware or software operation such as the interaction with peripheral components or devices.

Software 730 may include code to implement aspects of the present disclosure, including code to support alarm management. Software 730 may be stored in a non-transitory computer-readable medium such as system memory or other memory. In some cases, the software 730 may not be directly executable by the processor but may cause a computer (e.g., when compiled and executed) to perform functions described herein.

Transceiver 735 may communicate bi-directionally, via one or more antennas, wired, or wireless links as described above. For example, the transceiver 735 may represent a wireless transceiver and may communicate bi-directionally with another wireless transceiver. The transceiver 735 may also include a modem to modulate the packets and provide the modulated packets to the antennas for transmission, and to demodulate packets received from the antennas.

I/O controller 740 may manage input and output signals for device 705. I/O controller 740 may also manage peripherals not integrated into device 705. In some cases, I/O controller 740 may represent a physical connection or port to an external peripheral. In some cases, I/O controller 740 may utilize an operating system such as iOS®, ANDROID®, MS-DOS®, MS-WINDOWS®, OS/2®, UNIX®, LINUX®, or another known operating system. In other cases, I/O controller 740 may represent or interact with a modem, a keyboard, a mouse, a touchscreen, or a similar device. In some cases, I/O controller 740 may be implemented as part of a processor. In some cases, a user may interact with device 705 via I/O controller 740 or via hardware components controlled by I/O controller 740.

User interface 745 may enable a user to interact with device 705. In some embodiments, the user interface 745 may include an audio device, such as an external speaker system, an external display device such as a display screen, or an input device (e.g., remote control device interfaced with the user interface 745 directly or through the I/O controller module).

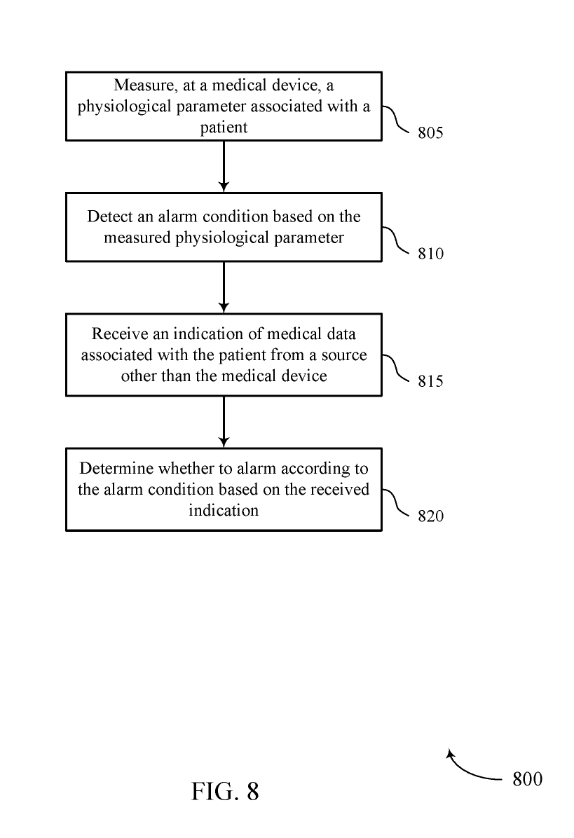

FIG. 8 shows a flowchart illustrating a method 800 for alarm management in accordance with aspects of the present disclosure. The operations of method 800 may be implemented by a medical device or its components as described herein. For example, the operations of method 800 may be performed by a collaborative alarm manager as described with reference to FIGS. 4 through 7. In some examples, a medical device may execute a set of codes to control the functional elements of the device to perform the functions described below. Additionally or alternatively, the medical device may perform aspects of the functions described below using special-purpose hardware.

At 805 the medical device may measure a physiological parameter associated with a patient. The operations of 805 may be performed according to the methods described herein. In certain examples, aspects of the operations of 805 may be performed by a physiological sensor as described with reference to FIGS. 4 through 7.

At 810 the medical device may detect an alarm condition based at least in part on the measured physiological parameter. The operations of 810 may be performed according to the methods described herein. In certain examples, aspects of the operations of 810 may be performed by an alarm component as described with reference to FIGS. 4 through 7.

At 815 the medical device may receive an indication of medical data associated with the patient from a source other than the medical device. The operations of 815 may be performed according to the methods described herein. In certain examples, aspects of the operations of 815 may be performed by an external medical data component as described with reference to FIGS. 4 through 7.

At 820 the medical device may determine whether to alarm according to the alarm condition based at least in part on the received indication. The operations of 820 may be performed according to the methods described herein. In certain examples, aspects of the operations of 820 may be performed by an alarm component as described with reference to FIGS. 4 through 7.

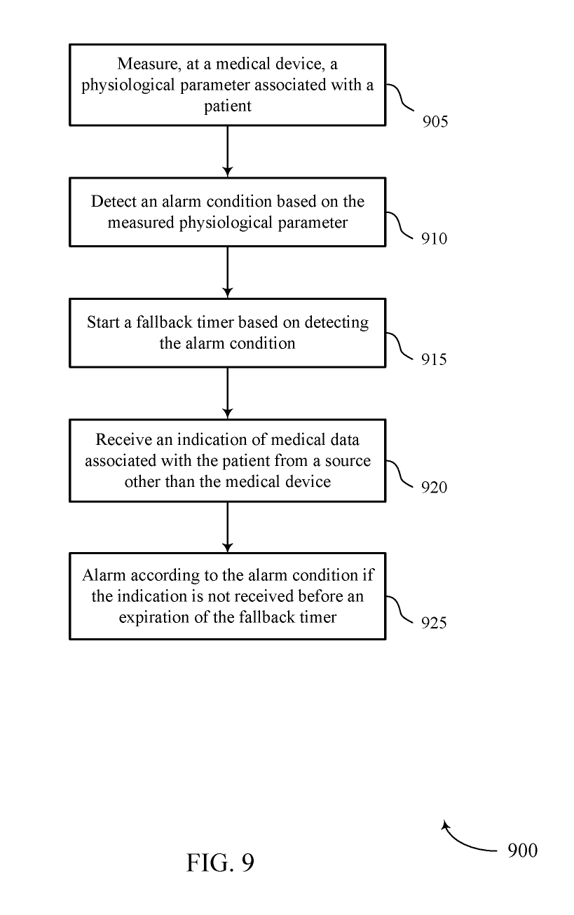

FIG. 9 shows a flowchart illustrating a method 900 for alarm management in accordance with aspects of the present disclosure. The operations of method 900 may be implemented by a medical device or its components as described herein. For example, the operations of method 900 may be performed by a collaborative alarm manager as described with reference to FIGS. 4 through 7. In some examples, a medical device may execute a set of codes to control the functional elements of the device to perform the functions described below. Additionally or alternatively, the medical device may perform aspects of the functions described below using special-purpose hardware.

At 905 the medical device may measure a physiological parameter associated with a patient. The operations of 905 may be performed according to the methods described herein. In certain examples, aspects of the operations of 905 may be performed by a physiological sensor as described with reference to FIGS. 4 through 7.

At 910 the medical device may detect an alarm condition based at least in part on the measured physiological parameter. The operations of 910 may be performed according to the methods described herein. In certain examples, aspects of the operations of 910 may be performed by an alarm component as described with reference to FIGS. 4 through 7.

At 915 the medical device may start a fallback timer based at least in part on detecting the alarm condition. The operations of 915 may be performed according to the methods described herein. In certain examples, aspects of the operations of 915 may be performed by a fallback timing component as described with reference to FIGS. 4 through 7.

At 920 the medical device may receive an indication of medical data associated with the patient from a source other than the medical device. The operations of 920 may be performed according to the methods described herein. In certain examples, aspects of the operations of 920 may be performed by an external medical data component as described with reference to FIGS. 4 through 7.

At 925 the medical device may alarm according to the alarm condition if the indication is not received before an expiration of the fallback timer. The operations of 925 may be performed according to the methods described herein. In certain examples, aspects of the operations of 925 may be performed by an alarm component as described with reference to FIGS. 4 through 7.

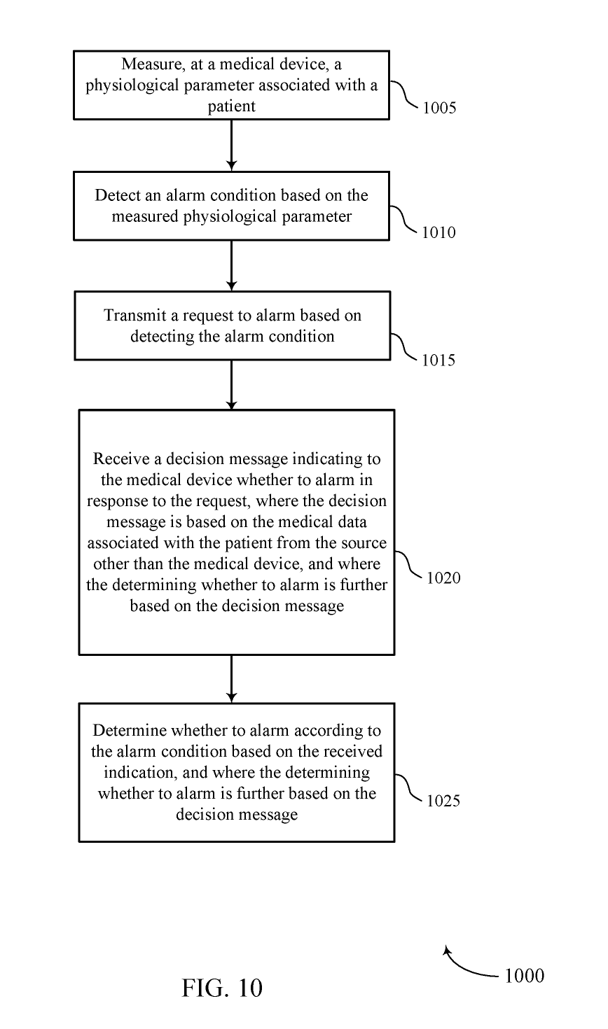

FIG. 10 shows a flowchart illustrating a method 1000 for alarm management in accordance with aspects of the present disclosure. The operations of method 1000 may be implemented by a medical device or its components as described herein. For example, the operations of method 1000 may be performed by a collaborative alarm manager as described with reference to FIGS. 4 through 7. In some examples, a medical device may execute a set of codes to control the functional elements of the device to perform the functions described below. Additionally or alternatively, the medical device may perform aspects of the functions described below using special-purpose hardware.

At 1005 the medical device may measure a physiological parameter associated with a patient. The operations of 1005 may be performed according to the methods described herein. In certain examples, aspects of the operations of 1005 may be performed by a physiological sensor as described with reference to FIGS. 4 through 7.

At 1010 the medical device may detect an alarm condition based at least in part on the measured physiological parameter. The operations of 1010 may be performed according to the methods described herein. In certain examples, aspects of the operations of 1010 may be performed by an alarm component as described with reference to FIGS. 4 through 7.

At 1015 the medical device may transmit a request to alarm based at least in part on detecting the alarm condition. The operations of 1015 may be performed according to the methods described herein. In certain examples, aspects of the operations of 1015 may be performed by an alarm request component as described with reference to FIGS. 4 through 7.

At 1020 the medical device may receive a decision message indicating to the medical device whether to alarm in response to the request, wherein the decision message is based at least in part on the medical data associated with the patient from the source other than the medical device, and wherein the determining whether to alarm is further based at least in part on the decision message. The operations of 1020 may be performed according to the methods described herein. In certain examples, aspects of the operations of 1020 may be performed by an alarm request component as described with reference to FIGS. 4 through 7.

At 1025 the medical device may determine whether to alarm according to the alarm condition based at least in part on the received indication, and where the determining whether to alarm is further based on the decision message. The operations of 1025 may be performed according to the methods described herein. In certain examples, aspects of the operations of 1025 may be performed by an alarm component as described with reference to FIGS. 4 through 7.

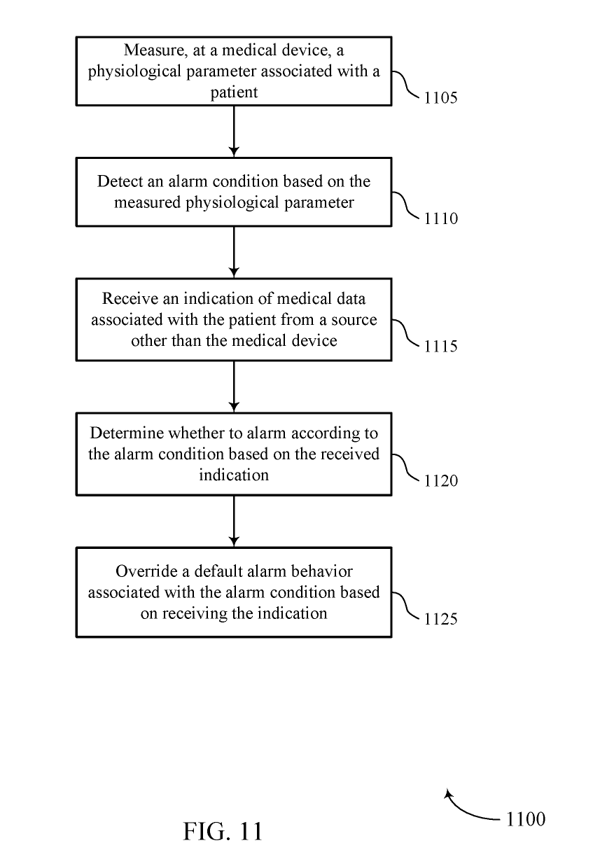

FIG. 11 shows a flowchart illustrating a method 1100 for alarm management in accordance with aspects of the present disclosure. The operations of method 1100 may be implemented by a medical device or its components as described herein. For example, the operations of method 1100 may be performed by a collaborative alarm manager as described with reference to FIGS. 4 through 7. In some examples, a medical device may execute a set of codes to control the functional elements of the device to perform the functions described below. Additionally or alternatively, the medical device may perform aspects of the functions described below using special-purpose hardware.

At 1105 the medical device may measure a physiological parameter associated with a patient. The operations of 1105 may be performed according to the methods described herein. In certain examples, aspects of the operations of 1105 may be performed by a physiological sensor as described with reference to FIGS. 4 through 7.

At 1110 the medical device may detect an alarm condition based at least in part on the measured physiological parameter. The operations of 1110 may be performed according to the methods described herein. In certain examples, aspects of the operations of 1110 may be performed by an alarm component as described with reference to FIGS. 4 through 7.

At 1115 the medical device may receive an indication of medical data associated with the patient from a source other than the medical device. The operations of 1115 may be performed according to the methods described herein. In certain examples, aspects of the operations of 1115 may be performed by an external medical data component as described with reference to FIGS. 4 through 7.

At 1120 the medical device may determine whether to alarm according to the alarm condition based at least in part on the received indication. The operations of 1120 may be performed according to the methods described herein. In certain examples, aspects of the operations of 1120 may be performed by an alarm component as described with reference to FIGS. 4 through 7.

At 1125 the medical device may override a default alarm behavior associated with the alarm condition based at least in part on receiving the indication. The operations of 1125 may be performed according to the methods described herein. In certain examples, aspects of the operations of 1125 may be performed by an alarm component as described with reference to FIGS. 4 through 7.

It should be noted that the methods described above describe possible implementations, and that the operations and the steps may be rearranged or otherwise modified and that other implementations are possible. Furthermore, aspects from two or more of the methods may be combined.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof

The various illustrative blocks and modules described in connection with the disclosure herein may be implemented or performed with a general-purpose processor, a digital signal processor (DSP), an ASIC, an field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration). A processor may in some cases be in electronic communication with a memory, where the memory stores instructions that are executable by the processor. Thus, the functions described herein may be performed by one or more other processing units (or cores), on at least one integrated circuit (IC). In various examples, different types of ICs may be used (e.g., Structured/Platform ASICs, an FPGA, or another semi-custom IC), which may be programmed in any manner known in the art. The functions of each unit may also be implemented, in whole or in part, with instructions embodied in a memory, formatted to be executed by one or more general or application-specific processors.

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above may be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media may comprise RAM, ROM, electrically erasable programmable read only memory (EEPROM), compact disk (CD) ROM or other optical disk storage, magnetic disk storage or other magnetic storage devices, or any other non-transitory medium that may be used to carry or store desired program code means in the form of instructions or data structures and that may be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, digital subscriber line (DSL), or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method for patient monitoring, comprising:
measuring, at a medical device, a physiological parameter associated with a patient;
detecting an alarm condition based at least in part on the measured physiological parameter;
receiving an indication of medical data associated with the patient from a source other than the medical device; and
determining whether to alarm according to the alarm condition based at least in part on the received indication.

2. The method of claim 1, further comprising:
starting a fallback timer based at least in part on detecting the alarm condition; and
alarming according to the alarm condition if the indication is not received before an expiration of the fallback timer.

3. The method of claim 1, further comprising:
transmitting a request to alarm based at least in part on detecting the alarm condition.

4. The method of claim 3, further comprising:
receiving a decision message indicating to the medical device whether to alarm in response to the request, wherein the decision message is based at least in part on the medical data associated with the patient from the source other than the medical device, and wherein the determining whether to alarm is further based at least in part on the decision message.

5. The method of claim 1, further comprising:
overriding a default alarm behavior associated with the alarm condition based at least in part on receiving the indication.

6. The method of claim 1, further comprising:
alarming based at least in part on receiving the indication.

7. The method of claim 1, further comprising:
transmitting a request for at least a subset of the medical data associated with the patient, wherein the indication of medical data is based at least in part on the request for at least the subset of the medical data.

8. The method of claim 1, wherein the source other than the medical device comprises one or more other medical devices, manually entered data, or both.

9. The method of claim 8, wherein the determining whether to alarm is based at least in part on a priority of the medical device with respect to a priority of the source other than the medical device.

10. The method of claim 1, wherein the alarm condition is a default alarm associated with the medical device.

11. A medical device for patient monitoring, comprising:
a processor;
memory in electronic communication with the processor; and
instructions stored in the memory and operable, when executed by the processor, to cause the medical device to:
measure, at the medical device, a physiological parameter associated with a patient;
detect an alarm condition based at least in part on the measured physiological parameter;
receive an indication of medical data associated with the patient from a source other than the medical device; and
determine whether to alarm according to the alarm condition based at least in part on the received indication.

12. The medical device of claim 11, wherein the instruction stored in the memory comprise instructions operable to cause the apparatus to:
start a fallback timer based at least in part on detecting the alarm condition; and
alarm according to the alarm condition if the indication is not received before an expiration of the fallback timer.

13. The medical device of claim 11, wherein the instruction stored in the memory comprise instructions operable to cause the apparatus to:
transmit a request to alarm based at least in part on detecting the alarm condition.

14. The medical device of claim 13, wherein the instruction stored in the memory comprise instructions operable to cause the apparatus to:
receive a decision message indicating to the medical device whether to alarm in response to the request, wherein the decision message is based at least in part on the medical data associated with the patient from the source other than the medical device, and wherein the determination of whether to alarm is further based at least in part on the decision message.

15. The medical device of claim 11, wherein the instruction stored in the memory comprise instructions operable to cause the apparatus to:
override a default alarm behavior associated with the alarm condition based at least in part on receiving the indication.

16. The medical device of claim 11, wherein the instruction stored in the memory comprise instructions operable to cause the apparatus to:
alarm based at least in part on receiving the indication.

17. The medical device of claim 11, wherein the instruction stored in the memory comprise instructions operable to cause the apparatus to:
transmit a request for at least a subset of the medical data associated with the patient, wherein the indication of medical data is based at least in part on the request for at least the subset of the medical data.

18. The medical device of claim 11, wherein the source other than the medical device comprises one or more other medical devices, manually entered data, or both.

19. The medical device of claim 11, wherein the alarm condition is a default alarm associated with the medical device.

20. A non-transitory computer readable medium storing code for patient monitoring, the code comprising instructions executable by a processor to:
measure, at a medical device, a physiological parameter associated with a patient;
detect an alarm condition based at least in part on the measured physiological parameter;
receive an indication of medical data associated with the patient from a source other than the medical device; and
determine whether to alarm according to the alarm condition based at least in part on the received indication.
